(19) United States
(12) Patent Application Publication
(10) Pub. No.: US 2014/0163520 A1
(43) Pub. Date: Jun. 12, 2014

(54) COLLAGEN BIOMATERIAL FOR CONTAINMENT OF BIOMATERIALS

(71) Applicant: OSSEOUS TECHNOLOGIES OF AMERICA, Newport Beach, CA (US)

(72) Inventors: Edwin SHORS, Laguna Beach, CA (US); William KNOX, Newport Beach, CA (US)

(73) Assignee: OSSEOUS TECHNOLOGIES OF AMERICA, Newport Beach, CA (US)

(21) Appl. No.: 14/092,642

(22) Filed: Nov. 27, 2013

Related U.S. Application Data
(63) Continuation of application No. 13/262,889, filed on Feb. 16, 2012.
(60) Provisional application No. 61/168,202, filed on Apr. 9, 2009.

(51) Int. Cl. A61B 17/88; A61F 2/30

(52) U.S. Cl. CPC A61B 17/8805; A61F 2/30723
USPC 604/506

(57) ABSTRACT

A biocompatible, resorbable collagen membrane containment member for a bone regenerative material, and a method of using such a containment member to regenerate a bone defect by surgically accessing the bone defect; disposing the containment member adjacent the bone defect, and thereafter injecting a bone regenerative material into the containment member.

### CROSS REFERENCE TO RELATED APPLICATIONS

This application is a Continuation of U.S. application Ser. No. 13/262,889 filed on Feb. 16, 2012. Application Ser. No. 13/262,889 is the National Phase of PCT International Application No. PCT/US2010/030046 filed on Apr. 6, 2010, which claims priority to U.S. Provisional Patent Application No. 61/168,202 filed on Apr. 9, 2009, all of which are hereby expressly incorporated by reference into the present application.

### FIELD OF THE INVENTION

The present invention relates to a malleable collagen membrane for containment of biomaterials within a bone cavity in a human or other mammal.

### BACKGROUND OF THE INVENTION

Bone is the body's primarily structural tissue; consequently it can fracture and biomechanically fail. Fortunately, it has a remarkable ability to regenerate because bone tissue contains stem cells which are stimulated to form new bone within bone tissue and adjacent to the existing bone. Boney defects regenerate from stem cells residing in viable bone, stimulated by signal proteins, and multiplying on existing cells or on an extracellular matrix (i.e., trellis). Like all tissues, bone requires support via the vascular system to supply nutrients and cells, and to remove waste. Bone will not regenerate without prompt regeneration of new blood vessels (i.e., neovascularization), typically with the first days and weeks of the regenerative cascade. Bone must also be stabilized during the healing process. Micro-motion or micro-strains are the forces and displacements invoked by the activities of daily living. They are imperceptible by touch or vision. In contrast, macromotion and macro-strains are detectable by touch or vision. Although micro-strains promote bone formation, macro-strains inhibit bone formation. Macromotion inhibits bone formation, in favor of either cartilage or soft tissue formation.

Various attempts have been made in the past to stimulate or augment bone regeneration by introducing a bone regenerating material proximate to a deteriorated bone structure. Bone regenerating materials are classified as “bioactive” because they are biocompatible and stimulate new bone formation. Examples of bioactive materials are autograft, osteogenic stem cells, osteoinductive proteins, and osteoconductive matrices. Bioactive agents are typically delivered to the operative site by the surgeon as deformable, flowable biomaterials. The predictability of bioactive agents is poor, however because surgeons have been unable to adequately control the placement of the bone regenerating material and thus guide the development of new or additional bone. Liquids, gels, granules, composites can be easily expressed from syringes into small defects but they can also go to unintended locations causing severe complications. Moreover, bioactive materials often migrate over time from the desired site. Thus, despite considerable efforts of the prior art, there has remained a long felt need for better methods of tissue augmentation, especially for bone regeneration or augmentation.

Polymethyl-methacrylate bone cement has been used in orthopedic surgery for more than fifty years as inert, strong cement. The components are mixed intra-operatively and injected within minutes by the surgeon into the bone site. Although polymethyl-methacrylate bone cement is biocompatible after it has set up, some of its components during the crosslinking phase are highly toxic.

Another problem with polymethyl-methacrylate bone cement is that it is highly exothermic. Excessive heat can kill bone cells. Consequently, these complications are mitigated if the surgeon waits to inject the cement until the bone cement has transitioned from the low viscosity to high viscosity. However, low viscosity cements more easily conducts through syringes, catheters and small bone defects. Therefore, high viscosity bone cement works well as filler materials in large bone defects.

As a structural material, bone cement is used to permanently fix metallic hip and knee prosthesis into the cancellous bone. The cement percolates into the open porosity of the bone and onto the surface of the metal, because it is initially low viscosity and injectable. During this phase, the low viscosity cement can be injected from a syringe or molded into place. Over several minutes, its viscosity increases as heat is released. The inert polymer then sets up to a solid, high compressive strength cement to hold the metallic prosthesis in place.

Over the past decade a new application for polymethyl-methacrylate cement has emerged, called osteoplasty, vertebroplasty or kyphoplasty because it permanently fixes bone fractures, typically of the spine. It permanently reinforces bone fractures or weak bone structures that are unlikely to regenerate with new bone. The principal indications are for reinforcing fractures of the vertebral body of the spine. To accomplish a vertebroplasty, the surgeon accesses the vertebral fracture by drilling into the fracture site, advancing a catheter into the drill hole, and forcing methacylate cement into the cancellous bone. The cement remains permanently to stabilize the vertebral body. It does not alter the anatomy of the fractured vertebra body because the cement fixes the structures in place permanently. This therapy often brings immediate relief of pain to the patient, which is the primary goal. Pain relief may be due to stabilization of the macromotion or by the exothermic heat of the methacylate cement.

Kyphoplasty is a variation on the vertebroplasty procedure to restore original anatomical positioning. A balloon catheter is inserted into the drill hole and the balloon is inflated to compress weak cancellous bone and to restore alignment. After the elastomeric balloon is removed, methacylate cement is injected. Although Kyphoplasty has become more common, complications of this procedure are numerous. For example, the low viscosity cement may migrate outside of the vertebral body where it can impinge on vital structures such as the spinal cord. In addition, the toxic monomers from the cement can enter the vascular system causing lethal toxic shock.

Conventional highly porous implantable collagen membranes typically have been made of reconstituted, reticulated bovine (i.e., cow) collagen. Such materials are conventionally provided to surgeons as rectilinear sheets with uniform thicknesses of approximately 1 mm. Their low density and high porosity make such materials supple and conformable. Unfortunately, however, they therefore also have a low tensile strength and stiffness, particularly after wetting with saline or blood, and are inadequate for use as a containment device in surgical applications. Rather, they are difficult to handle and liable to tear themselves. In addition, such materials are difficult to retain in a desired position because they are so thin and fragile that they are difficult to attach at the desired location with a bone tack or suture.

### SUMMARY OF THE INVENTION

The present invention provides a containment device for positioning, localizing and containing bioactive or inert biomaterials to the position desired. The device is a three dimensional collagen membrane which serves as a barrier or container. The collagen membrane is sufficiently tough and strong to contain and retain bioactive or bioinert biomaterials. The resorbable collagen three-dimensional membrane may be used by surgeons as an implantable medical device to aid in a variety of tissue regenerative indications.

The present invention provides a resorbable biomaterial for biomaterial containment. The collagen container may have uniform properties or it may have selected areas of higher strength, toughness and stiffness with other areas of lower strength, toughness and stiffness. The invention thus provides a biocompatible and resorbable collagen three dimensional membrane, for containment of bioactive and bioinert biomaterials which is ideal for many bone reconstructive indications.

The three-dimensional shaped collagen membranes of the invention serve three functions. First, they serve as a protective barrier that may prevent bioactive bone grafting materials or bioinert bone cement from flowing to undesired locations. Second, they serve as a biological trellis for tissue regeneration, particularly promoting regeneration of fibrovascular tissue to eventually resorb the container. The collagen is biocompatible and porous for ingrowth of connective tissue. Third, they serve as a structural barrier, allowing the clinician to more effectively localize bioactive or bioinert biomaterials.

Trellises of porous biomaterials (i.e., matrices) serve as a framework on which and through which tissue can grow. Most tissues, including bone and fibrovascular tissue, proliferate only by attaching to a structure or matrix. Cells then multiply and expand on pre-existing cells, extra-cellular matrix or biomaterials. Therefore, these matrices must have porosity. However, porosity generally decreases strength, typically non-linearly such that a small amount of porosity disproportionally decreases mechanical properties. The optimal porosity has been characterized in the musculoskeletal, field, for various principal regenerative tissues. For neovascular tissue (i.e., new blood vessels), pore diameters must be larger than 20 micrometers. For osteoid (non-mineralized bone), pore diameters must be larger than 50 micrometers. For bone formation, pore diameters must be larger than 100 micrometers.

Assuring precise positioning of implanted tissue augmentation materials in a living body can be a difficult task. Moreover, because a living body is a dynamic environment, implanted materials may shift in position over time. The use of strategically shaped and implanted membranes according to the present invention, however, facilitates precise placement of implanted biomaterials and enables containment or retention of the implanted biomaterial at the desired location within the body.

The present invention makes use of collagen as a resorbable biomaterial for implantable medical devices to aid in tissue regeneration and repair. Depending on the extent of cross linking, collagen biomaterials can be manufactured to resorb over a prescribed range, typically from a few weeks to one year.

The present invention uses collagen membranes having a three dimensional shape to contain bioactive bone grafting materials and/or bioinert bone cements. It also facilitates tissue regeneration, particularly bone and fibrovascular tissue. This bioresorbable collagen containment member can be manufactured by casting collagen between molds which form a three dimensional shape between them and lyophilizing, to form a highly porous structure. The resulting three dimensionally shaped collagen membranes are then moistened and dried. This process increases the density and cross linking to provide high strength, high stiffness membranes which are nevertheless sufficiently malleable to be formed into a desired configuration to fit a surgical site in order to support a tissue membrane and/or retain surgically introduced bone graft material in a desired location.

The three dimensionally shaped collagen membranes of the invention can be manufactured by casting process using molds which form a three dimensionally shaped mold cavity between them. The mold cavity is filled with a collagen suspension. After lyophilization, the mold is opened and the resulting collagen membrane containment member removed. The membrane can then be rehydrated and dried to provide a high strength three dimensional form.

If desired, macroscopic holes can be made in the membrane with strategically placed pins transecting the mold cavity which are removed before the mold is opened. Alternatively, macroscopic holes can then be made in the membrane after rehydration and drying with strategically placed pins, cuts, or laser cutting. In yet another alternative, the membrane may be made by a selective rehydration/drying process in which a selected portion of the membrane is rehydrated and dried to provide a high strength three dimensional form while the remaining portion that is not rehydrated/dried retains an open porosity, but has a lower strength and stiffness.

The three dimensionally shaped collagen membrane of the invention has a number of important advantages for biomaterial containment. Thinner portions of the membrane exhibit optimal porosity to assure neovascular ingrowth and bone cell ingrowth because pores of the required dimensions are precisely manufactured.

The three dimensionally shaped collagen membrane of the invention also exhibits optimal strength. The membrane of the invention assures that the optimal mechanical properties are provided in collagen membranes so that they can be formed by bending and/or cutting to a desired configuration to match an intended surgical site and afterward will retain that configuration under normal loading conditions.

The thickness of the three dimensional collagen implants can be adjusted for biological, mechanical or intra-operative handling advantages. Thickness can alter the resorption rate of the membrane. It can also alter the strength of the membrane, thus modifying the resistance to forces applied by the bioactive or bioinert materials forced into the device. Also, varying the thickness can assist the clinician to locate the device intra-operatively. As, an example, thicker membranes improve user-friendliness for the surgeon by making it easier for the surgeon to identify the proper orientation of the membrane and also by facilitating handling. Because the thicker portions exhibit stronger mechanical properties, such as tensile strength or tear strength, due to its larger cross-sectional area, the collagen membrane containment member exhibits greatly improved resistance to tearing.

Collagen membranes in the form of balloons are not highly elastic. Therefore, the surface area does not change with internal pressure. Therefore, collagen balloons do not inherently collapse. For some applications, it is necessary to insert the collagen three-dimensional membrane structure into the surgical site through a narrow cannula. In the present invention, the three dimensional balloons or capsules can be folded, similar to an accordion or bellows. This allows the collagen membrane structures to be inserted through a cannula. Compaction of the collagen membrane device can be accomplished by incorporating fold in the membrane. Folding can accomplished using adjoining and alternative thin and thick membranes. Folding can also be accomplished using thin and thick membranes in tandem to provide creases for folding.

The thickness of the thicker may range from about 1 mm to about 5 mm, preferably about 1.5 mm to about 3.5 mm, and particularly preferably about 2 mm. The transition between the thick portions and the thinner portions may be linear, or in other words, the collagen membrane containment member may have a uniform taper from the thick portion to the thinner portion, thereby giving rise to a smooth surface. Alternatively, the transition between the thick portions and the thinner portions may be a step function; giving rise to a membrane comprised of adjacent sections each having a progressively smaller thickness.

Thinner portions of the collagen membrane containment member provide a collagen membrane that is simultaneously both malleable and resiliently elastic. By malleable is meant that the membrane can be folded to a desired shape or configuration and then will retain that configuration. This is achieved by bending the membrane beyond the elastic limit of the material and then creasing the membrane at the bending site. As a result, the membrane will retain its shape after being custom bent, intra-operatively by the surgeon.

By resiliently elastic is meant that the membrane is semi-rigid but will readily deform when pressed into contact with the surgical site so as to conform to the configuration of the surgical site. At the same time it resists permanent shape change so that restoring forces in the membrane will urge the membrane to reassume its original configuration, thereby biasing the membrane against the surgical site. This is achieved insofar as the elastic limit of the membrane is not exceeded so that no permanent deformation arises.

The thickness of the thinner portions may range from about 0.3 mm to about 1.5 mm, preferably from about 0.4 mm to about 1.0 mm, and particularly preferably about 0.5 mm.

The collagen membrane may also be easily trimmed by scissors or scalpel to fit the surgical site. It is preferred to trim the membrane to slightly oversize dimensions so that a snug fit will be generated due to the resilient elasticity of the membrane.

This combination of malleability and resilient elasticity results in a membrane which is readily formable and bendable by the surgeon to fit the surgical site and which provides a snug fit to assure positional stability of the membrane and also effective retention of bone graft material in the desired location.

As used herein, the term “lyophilization” refers to “freeze drying” or vacuum drying.

In the process for producing the membranes of the invention, the molded collagen suspension is placed in a freezer and then a vacuum is applied. Under vacuum, the water within the collagen moves directly from the solid phase to the gas phase. Consequently, there is no shrinking or change to the dimensions. This makes a highly porous, but relatively weak collagen structure. A key step in the production process according to the invention is then to lightly wet the porous collagen with alcohol/water which collapses the porosity. The material is then air dried. This makes a much stronger/stiffer collagen membrane. Air drying at elevated temperatures also cross-links some of the collagen molecules to further increase the strength and decrease the resorption rate.

### BRIEF DESCRIPTION OF THE FIGURES

The invention will be described in further detail hereinafter with reference to illustrative examples of preferred embodiments shown in the accompanying drawing figures in which:

FIGS. 1 and 2 are perspective views of a densified collagen biomaterial membrane containment member according to the invention; FIG. 1A: side view denoting cavity wall; FIG. 1B: frontal view looking into cavity; FIG. 2A: side view denoting cavity wall; FIG. 2B: frontal view looking into cavity; FIG. 2C: view from back;

FIGS. 3-5 are schematic views of the collagen material as used to repair a bone defect site, FIG. 3: the capsule in place, FIG. 4: the capsule in place and either a bioactive bone grafting materials or a bioinert cement placed into the collagen capsule; FIG. 5: the capsule in place and either a bioactive bone grafting material or a bioinert cement placed into the capsule, with a portion of the capsule being selectively permeable to the biomaterial; and

FIG. 6 is a schematic representation of a foldable version of the collagen membrane containment member of the invention.

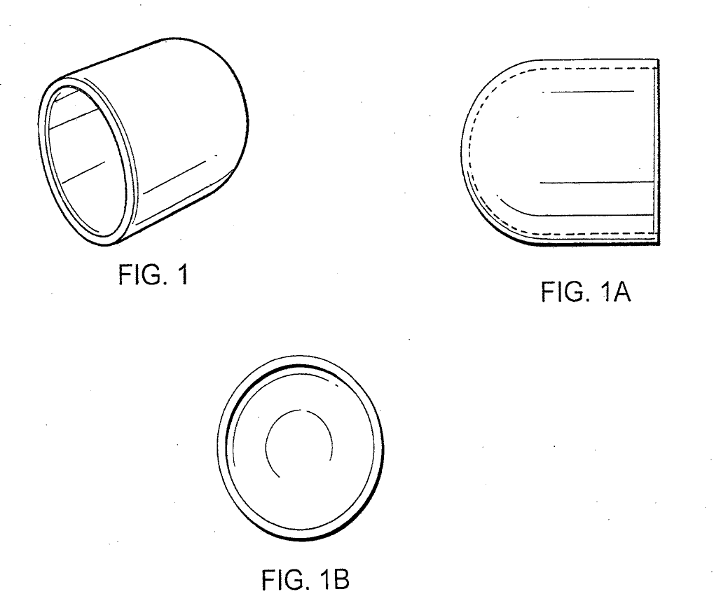

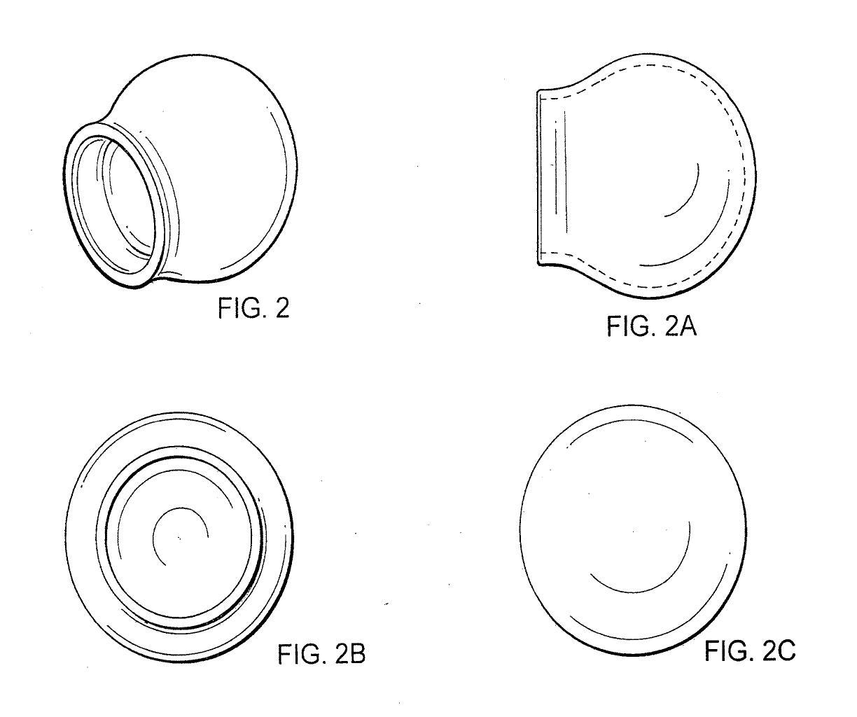

### DETAILED DESCRIPTION OF THE INVENTION

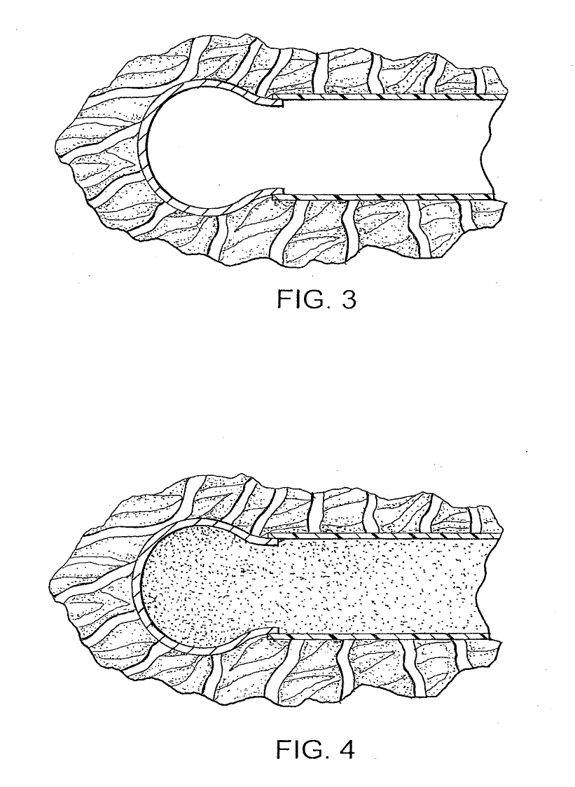

The bone defect site may be formed spontaneously; it may be caused by traumatic fracture, or it may be intentionally produced by the surgeon, using a balloon catheter or some other such device designed to make a void in bone. As shown in FIG. 3, the collagen membrane containment member has a generally balloon or capsular configuration, but it should be understood that the membrane could as well have other shapes including an oval, flat or generally triangular configuration.

The membrane can be easily trimmed during surgery with scissors or a scalpel for a custom fit to the surgical site.

The membrane need not be wetted prior to implantation, but can be wetted in place with saline or blood from the surgical site.

The three-dimensional membrane containment member can be purposefully designed and manufactured during the manufacturing process to the exact dimensions of the bone cavity or it can be bent to a desired configuration to fit the surgical site and generally has sufficient rigidity to retain the desired configuration so that it can retain implanted bone graft material in the desired location.

FIG. 3 shows the capsule in place. Initially, the surgeon has inserted a cannula into the cancellous bone site. The cannula may be inserted into a pre-existing bone cavity. Alternatively, the surgeon may create a bone cavity using a variety of existing surgical instruments, such as a balloon catheter. The balloon catheter can compress cancellous bone or it can reduce a fracture to its anatomic position, thereby creating a defect. The three-dimensional membrane, in this case illustrated as a sphere, is inserted though the catheter and spontaneously opened via hydration using blood or saline or local fluids. In some cases, the three-dimensional membrane will open up or expand by filling the internal volume with a fluid or biomaterial. In this illustration, the three-dimensional membrane has expanded to the surface of the bone.

FIG. 4 show the capsule in place and either a bioactive bone grafting materials or a bioinert cement placed into the collagen capsule. In this variation of the device, the capsule is manufactured to prevent any leak of the biomaterial through the membrane and into the porosity of the surrounding bone.

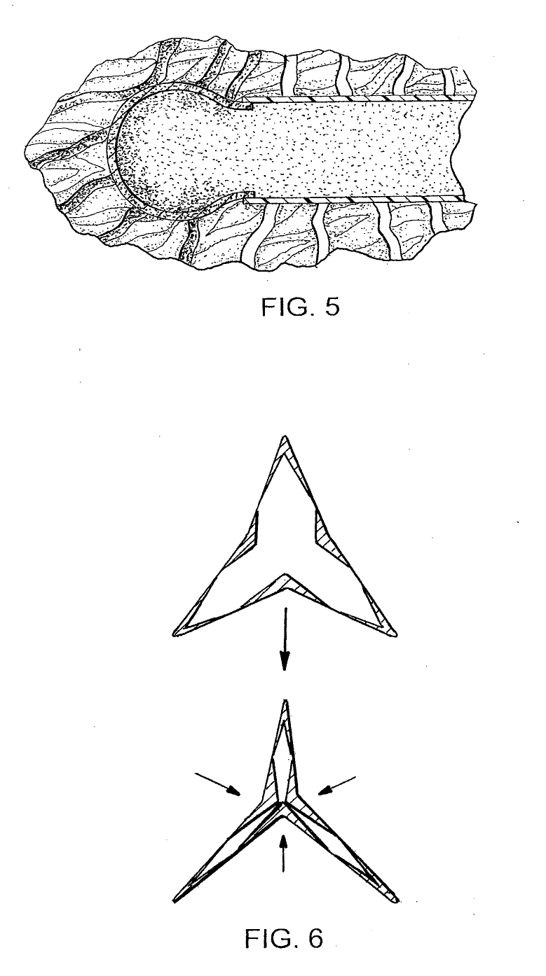

FIG. 5 shows the capsule in place and either a bioactive bone grafting material or a bioinert cement placed into the capsule. In this variant of the invention, the capsule is manufactured with a portion selectively permeable to the biomaterial. Therefore, the biomaterial, either the bioactive bone grafting materials or the bioinert cement, can leak selectively into some of the porosity of the surrounding bone.

FIG. 6 shows a version of the three dimensional collagen membrane that can be compressed into a smaller volume by alternative areas of compliance, called “nodes” in the figure. In this case, the nodes are produced by manufacturing the collagen to have more porosity and thickness. This decreases the rigidity at the nodes, allowing the thinner membranes to compress. With a smaller volume, the device can be more easily inserted through a surgical cannula and located within the bone cavity. The nodes will expand under pressure from the injection of the bioactive or bioinert biomaterial. The nodes can be made by adjusting the thickness/porosity of the membrane, but they can also be made by forming creases in the thinner membranes.

Collagen membrane is preferably distributed in a sterile package.

The three dimensional collagen biomaterial membrane of the invention can be produced as follows. A suspension of purified collagen is made in water/alcohol. The collagen is preferably in native fibrous form with a fiber length of from about 0.2 to 3 mm, preferably about 1.5 mm. The suspension advantageously may contain from about 10 to about 60 mg of collagen per ml of suspension, particularly preferably from about 15 to about 20 mg collagen per ml. The suspending medium may advantageously comprise from about 5% to about 25% ethanol in water, particularly preferably about 10% ethanol.

After de-aeration of the collagen suspension, the suspension is filled into a mold made up of two mold plates. The thickness of the resultant membrane can be modified by adjusting the gap between the two molds. The filled mold is then placed in a freezer at a temperature sufficient to solidify the suspension, e.g., −70° C. Once the suspension is solidified, the plates are separated, with the frozen collagen membrane containment member remaining on one of the plates.

The mold plate with the collagen membrane is then transferred to a freeze dryer and freeze dried. The freeze-dried collagen membrane containment member is then removed from the freeze dryer. The dried collagen is sprayed with an alcohol solution. Preferably the alcohol solution may contain about 40 to about 70% alcohol in water, particularly preferably about 50% ethanol in water. The collagen membrane containment member is then subject to air drying followed by vacuum drying until completely dry. Thereafter, the dried collagen membrane containment member is subjected to heat treatment at from about 100 to about 140° C. for from about 15 minutes to about 2 hours to cure the membrane. Particularly preferably the membrane is cured for about one-half hour at a temperature of approximately 130° C.

After curing, the collagen membrane containment member may be cut to desired size and sterilely packaged for distribution and use.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method of repairing a bone defect comprising: surgically accessing the bone defect; disposing a resorbable collagen membrane containment member adjacent the bone defect site; and injecting a bone cement or bone regenerative material into said containment member.

2. A method as claimed in claim 1, wherein the injected material is a bioactive bone regenerating material selected from the group consisting of autograft, osteogenic stem cells, osteoinductive proteins and osteoconductive matrices.

3. A method as claimed in claim 1, wherein said containment member has a substantially uniform thickness and tensile strength.

4. A method as claimed in claim 1, wherein said containment member has a variable thickness with areas of greater thickness and rigidity and areas of lesser thickness and greater flexibility.

5. A method as claimed in claim 4, wherein said containment member is inserted in a folded state and then opened in place adjacent the bone defect.

6. A method as claimed in claim 5, wherein said containment member is inserted through a cannula.

7. A method as claimed in claim 1, wherein a cavity is formed adjacent the bone defect and the containment member is thereafter inserted into the cavity.

8. A method as claimed in claim 1, wherein the injected material is an inert bone cement.

9. A method as claimed in claim 1, wherein said containment member is in the form of a capsule.
